(19) United States
(12) Patent Application Publication
(10) Pub. No.: US 2010/0209519 A1
(43) Pub. Date: Aug. 19, 2010

(54) PHARMACEUTICAL COMPOSITION FOR INHALATION DELIVERY AND FABRICATION METHOD THEREOF

(75) Inventors: Feng-Huei LIN, Taipei City (TW); King-Jen CHANG, Taipei City (TW); Ching-Li TSENG, Taipei City (TW); Chung-Hung CHENG, Taipei City (TW)

Correspondence Address: QUINTERO LAW OFFICE, PC, 615 Hampton Dr, Suite A202, Venice, CA, 90291, US

(73) Assignee: NATIONAL TAIWAN UNIVERSITY, Taipei (TW)

(21) Appl. No.: 12/480,626

(22) Filed: Jun. 8, 2009

(30) Foreign Application Priority Data
Feb. 16, 2009 (TW) TW98104802

(51) Int. Cl. A61K 9/51; A61K 33/24; A61P 35/00

(52) U.S. Cl. 424/492; 424/649

(57) ABSTRACT

A pharmaceutical composition for inhalation delivery is provided, including drugs and a gelatin nanoparticle encapsulating the drugs to form a drug-gelatin nanocomplex, wherein the surface of the gelatin nanoparticle is modified with cell-targeting molecules. A method for fabricating the pharmaceutical composition for inhalation delivery is also provided.

### CROSS REFERENCE TO RELATED APPLICATIONS

This application claims the benefit of Taiwan Application No. 098104802, filed Feb. 16, 2009.

### BACKGROUND OF THE INVENTION

1. Field of the Invention

The present invention relates to a pharmaceutical composition for targeting cells and fabrication method thereof and, in particular, relates to a pharmaceutical composition for inhalation delivery to target cells and fabrication method thereof.

2. Description of the Related Art

Lung cancer accounts for about 20% of all cancer deaths in Taiwan. It has been reported, that in 2004, about 500,000 cancer patients in the U.S., U.K., France, Germany, Spain, Italy and Japan were diagnosed with non-small cell lung cancer. It is assumed, that as air pollution increases, so does the number of cases of lung cancer patients.

In addition to lung cancer, the number of patients with respiratory diseases, such as asthma, chronic obstructive pulmonary disease or chronic bronchitis, has also increased along with increasing industrialization and air pollution. The drug treatment of lung diseases by oral, subcutaneous or intravenous administration, causes systematic response for patients. Side effects such as hair loss, rash, nausea, vomiting, diarrhea, allergic reactions, nephrotoxicity or neurotoxicity, etc. occur, as most drug treatments also negatively influence non-cancerous cells. Examples of drugs used for treatment of lung diseases include, Cisplatin or Carboplatin for lung cancer chemotherapy, wherein the growth of cancer cells are inhibited by blocking covalent bonds between DNA molecules within cells. Additionally, Navelbine, Taxol or Taxotere may be used to suppress production of cancer cells by preventing the formation of microtubules during cell division.

To improve drug treatments of lung cancer patients, nano-pharmaceutical compositions with conventional drugs have been disclosed. Advantages thereof include drug protection against the gastrointestinal tract, prolonged release time, increased absorption surface, and greater accumulation at the target area. Disclosed materials for encapsulating drugs include poly(lactides) (see, eg., U.S. Pat. No. 3,773,919), poly(lactide-co-glycolides) (see, eg., U.S. Pat. No. 4,767,628), polyethylene glycol (PEG) (see eg., U.S. Pat. No. 5,648,095) or glycoside (see, eg., U.S. Pat. No. 7,465,753).

Due to small size, nanoparticles used for inhalation delivery are more effective and direct than oral administration for the drug treatments of lung cancer. Advantages of the inhalation method include non-invasion, instant effect and simplicity in use for homecare. However, disadvantages include particle aggregation, drug attachment onto upper respiratory tracts, expellant by the bronchi cilia, and failure to reach the pulmonary alveoli and lung tissues. Thus, combinations using nanoparticles with drugs have been disclosed. U.S. Pat. No. 5,049,388 discloses aerosol droplets containing liposome and liposome-drug; U.S. Pat. No. 6,090,407 discloses small particle liposomes encapsulating active drugs by di-lauroyl-phosphatidylcholin (DLPC); and U.S. Pat. No. 6,468,798B1 discloses liposome-nucleic acid complexes delivered by aerosol.

The invention provides a nano-level pharmaceutical composition for targeting cells. The composition can increase the amount of unit dosage of drugs that reach targeted cells.

### BRIEF SUMMARY OF THE INVENTION

The invention provides a pharmaceutical composition for inhalation delivery, comprising drugs and a gelatin nanoparticle encapsulating the drugs to form a drug-gelatin nanocomplex, wherein the surface of the gelatin nanoparticle is modified with cell-targeting molecules.

The invention also provides a method for fabricating a pharmaceutical composition for inhalation delivery, comprising: providing a drug; adding the drug into a gelatin solution to form a gelatin nanoparticle encapsulating drug; and linking cell-targeting molecules on the surface of the gelatin nanoparticle for modification, wherein the drug and the gelatin nanoparticle form a drug-gelatin nanocomplex.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 is a schematic drawing of EGF(ligand)-modified gelatin nanoparticles (GP-Av-bEGF).

FIG. 2 shows a core-shell structure of the GP-Av-bEGF under transmission electron microscopy.

FIG. 3 shows aerosol size distribution of the GP-Av-bEGF after nebulization.

FIG. 4 shows cellular accumulation of the GP-Av and GP-Av-bEGF in A549, H520 and HFL1 cell lines.

FIG. 5 shows in vivo fluorescent images for GP-Av and GP-Av-bEGF.

FIG. 5(a) is a PBS-treating group;

FIG. 5(b) is a GP-Av-treating group; and

FIG. 5(c) is a GP-Av-bEGF-treating group.

FIG. 6 shows relative fluorescence of the GP-Av-bEGF in organs after administration to A549-induced mice at 0.5 hours.

FIG. 7 shows relative fluorescence of the GP-Av-bEGF in organs after administration to A549-induced mice at 24 hours.

FIG. 8 shows Pt concentration in plasma, lungs and kidneys after in vivo administrating CDDP, GP-CDDP and GP-CDDP-bEGF at 0.5, 24 and 48 hours.

FIG. 8(a) shows Pt concentration in plasma;

FIG. 8(b) shows Pt concentration in lungs; and

FIG. 8(c) shows Pt concentration in kidneys.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

The pharmaceutical composition of the invention comprises drugs and a gelatin nanoparticle encapsulating the drugs to form a drug-gelatin nanocomplex. Gelatin is biodegradable, bio-compatible, plastic, and has adhesive properties (Pappas N A., et al., An Introduction to Materials in Medicine. Biomaterials Science. New York: Academic Press: 1996. p. 60-64), which is applicable in the invention. Gelatin used in the invention has 20,000-100,000 of Molecular Weight. In one embodiment of the invention, gelatin forms an aqueous solution with a concentration of 0.1-10% (weight-to-volume ratio), more preferably with a concentration of 1-6% (weight-to-volume ratio). In one embodiment, the gelatin solution forms nanoparticles at a room temperature of 50° C., a pH of 2-4. The obtained nanoparticles have approximate radius of 150-300 nm, without encapsulation of drugs.

Gelatin is known to be non-toxic, and is easily degraded by enzymes. In one embodiment of the invention, the gelatin solution further comprises a cross linker for enforcing the mechanic strength of the gelatin against enzymatic degradation without destroying bio-compatibility and biodegradability thereof. The cross linker may be selected from the group consisted of formaldehyde, glutaraldehyde, genipin, and diadehyde starch (DAS), but is not limited thereto. One embodiment of the cross linker is 0.01-0.1% in weight based on the weight of gelatin. In one embodiment, the cross-linking degree between the gelatin and the cross linker is preferably 1-20%.

The gelatin nanoparticles encapsulate drugs to form drug-gelatin complexes. Carboxyl, hydroxyl and amino groups are present on the surface of the drug-gelatin nanocomplex. The functional groups may link to cell-targeting molecules which functionalize as ligands. When the cell-targeting molecules bind to the receptors of target cells, the nanoparticles can specifically attach to the cell. Through receptor-mediated endocytosis, the nanoparticles are swallowed by the cell, and the drugs encapsulated in the particles are directly delivered to the cells. The drug-gelatin nanocomplex, therefore, target cells and efficiently deliver drugs to affected areas.

The cell-targeting molecules used in the invention may be designed according to the target cells or tissues, such as cancerous cells with specific receptor expressions. The cell-targeting molecules may include, for example, epithelial growth factor (EGF), anti-epithelial growth factor receptor (EGFR) antibody, anti-EGFR peptide or EGFR inhibitors for non-small cell lung cancer; anti-human epidermal growth factor receptor 2 (HER2) antibody, anti-HER2 peptide or HER2 inhibitors for breast cancer; folic acid or folic acid analogy for non-small cell lung cancer or malignant pleural mesothelioma; vascular epithelial growth factor (VEGF), anti-vascular epithelial growth factor receptor (VEGFR) antibodies, anti-VEGFR peptide or VEGFR inhibitor for colorectal cancer, renal cell cancer, breast cancer or ovarian cancer, and insulin-like growth factors (IGFs), anti-IGFs receptor (IGFR) antibody, anti-IGFR peptide or IGFR inhibitor. Any developed or undeveloped molecule that can identify (or bind to) specific cells may be used as the ligand in the invention.

The linkage between the cell-targeting molecules and the nanoparticles may be a chemical or biological linkage using specific molecules. The specific molecules may be avidin and biotin, antigen and antibodies, or cell markers, and are not limited thereto. One embodiment of the invention uses avidin and biotin for conjugation.

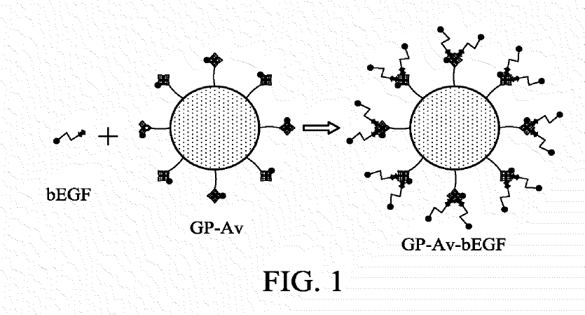

Avidin and biotin have four conjugated points which is stable and unaffected by different temperatures or a pH of values. In one embodiment of the invention, the surface of the gelatin nanoparticle is modified as sulfhydryl groups from amino groups. Next, avidin is conjugated with the sulfhydryl groups to form the avidin-modified gelatin nanoparticle (GP-Av), as shown in FIG. 1.

Biotinylation reagents may also react with the functional groups (—NH₂ or —COOH) of the cell-targeting molecules. One end of the biotinylation reagent binds biotin and the other end binds a cross-linker that chemically reacts with functional groups of bio-molecules (nucleic acid or protein). In one embodiment of the invention, EGF is modified as biotinylated EGF (bEGF), conjugated with GP-Av by the conjugation of avidin and biotin, to form a nanoparticle of the invention. Note that biotin is suitable to be used to bind onto different bio-molecules due to its relatively small size and fast and strong binding ability with avidin.

In one embodiment, avidin and biotin can be modified at a room temperature to 4° C., with a pH of 7-8. The specific molecules preferably have a molar ratio of 4:1 to 30:1 to the cell-targeting molecules, as reaction is minimal for molar ratios below 4:1 and excess unbinding continues to occur if the molar ratio is more than 30:1.

A fluorescent substrate may be used to bind with the specific molecules for easier observation of the nanocomplex. The fluorescent substrate may include substrates such as, flourescein isothiocyanate (FITC); tetramethyl rodamine (TRITC); aminomethyl coumarin (AMCA); or 4-methylumbelliferone (4-Men), but are not limited thereto. Fluorescent substrates are selected according to the size, properties and specificity of the specific molecules and observation devices.

In one embodiment of the invention, the pharmaceutical composition is nebulized by a nebulizer. It has been known that aerosol gelatin particles have aerodynamic stability (Deaton A T, et al., Generation of gelatin aerosol particles from nebulizer solutions as model drug carrier systems. Pharm Dev Technol 2002; 7:147-53; Morimoto et al., Gelatin microspheres as a pulmonary delivery system: evaluation of salmon calcitonin absorption. J Pharm Pharmacol 2000; 52:611-7). When the radius of the aerosol particle is more than 10 μm, the particle deposits on the upper respiratory tract and is removed by a lung defense mechanism, such as Mucociliary Clearance (MCC) and coughing, and fails to reach lung tissues (Knowles M, et al., Mucus clearance as a primary innate defense mechanism for mammalian airways. J Clin Invest 2002, 109(5):571-7). When the particle is between 1-5 μm, it will enter the trachea or bronchi. Particles with 0.5-1 μm in radius can enter the pulmonary alveolus after passing through the nasal, pharynx, trachea and bronchi systems. Meanwhile, particles with less than 0.5 μm in radius are expelled by exhalation.

The pharmaceutical composition of the invention is nebulized as aerosol droplets with 0.1-5 μm in radius. The size of the aerosol droplets can efficiently enter the lower respiratory tract without aggregation to the upper tract or being expelled by the lung defense system. When the aerosol droplets of the invention strike the wall of the tract or alveolus, they break and release the nanocomplex which later reaches the lung tissues. Meanwhile, note that direct delivery without nebulization would not be successful and the nanocomplex would be discarded while breathing.

The drugs of the invention are not limited, preferably hydrophilic drugs, for example: anticancer drugs, such as 5-Fluorouracil (5-FU), Cisplatin, Carboplatin, Gemcitabine, Vinorelbine, Paclitaxel, Paclitaxel analogue (ex. Docetaxel), or Doxorubin, etc.; drugs for asthma, such as steroids; bronchodilators, such as Albuterol, Salbutamol, or Salmeterol, etc.; anti-choline drugs, such as Ipratropium, or other drugs for respiratory or lung diseases. Any developed or experimental medicament may be used. In one embodiment of the invention, an anticancer drug, Cisplatin, is used. The drug is preferably 1-60% in weight based on the weight of the gelatin nanoparticle.

The pharmaceutical composition of the invention may be administrated alone or combined with conventional pharmaceutical acceptable carriers and/or excipients in an adequate proportion packed in a nebulizer. The dosage administered may be according to sex, age or body weight of patients and the condition of the disease or other factors.

The pharmaceutical composition of the invention targets affected lung tissues by using the nanocomplex with modification of cell-targeting molecules, wherein aerosol droplets thereof directly and efficiently reach affected lung tissues. Thus, providing a safe and effective means to administer treatment for respiratory and lung diseases.

### EXAMPLE 1

### Preparation of Gelatin Nanoparticles (GP)

Gelatin was derived from porcine skin (bloom 175) and dissolved in deionized water to form a 5% (w/v) aqueous gelatin solution. 5 ml of the solution was heated to 50° C. and subsequently added 5 ml of acetone. A precipitation was observed. The supernatant was discarded and the precipitate was resolved in deionized water again at 50° C. 12 ml of acetone was then added into the resolved gelatin solution at a pH of 2.5. Afterward, 0.04% of glutaraldehyde as a cross-linker was added and stirred at 1000 rpm overnight to form cross-linked gelatin nanoparticles. Finally, acetone was removed by vacuum dried. The GPs were suspended in deionized water and stored at 4° C. for further application.

### EXAMPLE 2

### NeutrAvidin^{FITC} Conjugation at the Surface of the GPs (GP-Av)

Initially, the GPs in deionized water were placed in a dialysis membrane bag and dialyzed against a sodium phosphate buffer containing 10 mM of EDTA (pH 8.0). 1 ml of the GPs solution (8 mg/ml) was reacted with 2-iminothiolane (28 mM) for 1 hour at 37° C. The GPs were thiolated, forming thiol groups on its surface. The thiolated GPs were centrifuged and purified in Amicon Ultra-4 filter devices (Millipore, USA) (Mw cutoff, 30,000). The thiol groups were spectrophotometrically determined by using the 5,5′-dithiol-bis-(2-nitrobenzoic acid) (DTNB) method.

Separately, NeutrAvidin^{FITC} (NeutrAvidin™) was dissolved in a sodium phosphate buffer (pH 7.2) containing 2 mg/ml of m-maleimidobenzoyl-N-hydroxysulfosuccinimide ester (Sulfo-MBS). The solution was well mixed at room temperature and reacted for 1 hour. The activated NeutrAvidin^{FITC} was purified on a gel filtration column.

The activated NeutrAvidin^{FITC} solution was then mixed with the thiolated nanoparticles and allowed to react overnight at 4° C. Activated NeutrAvidin^{FITC}-GP-conjugated nanoparticles (GP-Av) were obtained. After removing unbound NeutrAvidin^{FITC} derivatives. The GP-Av was concentrated by centrifugation using Amicon Ultra-4 filter devices (Millipore, USA) (Mw cutoff, 100,000).

### EXAMPLE 3

### Conjugation of Biotinylated EGF and NeutrAvidin^{FITC}-GPs

The EGF was initially dissolved in a phosphate-buffered saline (PBS, pH 7.0) and then added to a biotinylation reagent (Sulfo-NHS-LC-biotin)(Pierce, USA). The molar ratio of the Sulfo-NHS-LC-biotin to EGF was 15:1. The solution was mixed and left to react at room temperature for 30 min. Biotinylated EGF was separated by size exclusion chromatography by a D-slat dextran desalting column (Pierce, USA). The biotinylated EGF-containing elute fractions were collected, and the protein concentration of the biotinylated EGF was measured by using a bicinchoninic acid (BCA) protein assay kit (Sigma). The molar ratio of biotin binding with the EGF was determined using an EZ™ Biotin Quantitation kit (Pierce, USA).

250 μl bEGF (300 μg/ml) was mixed with 500 μl (4 mg/ml) of the GP-Av nanoparticles and incubated at 4° C. for 2 hours. The bEGF-conjugated GPs were washed with PBS and collected by centrifugation to purify the nanoparticles. These nanoparticles were abbreviated as GP-Av-bEGF.

The particle size of GPs and GP-Av-bEGF were determined by a photon correlation spectroscopy using an N4 Plus Submicron Particles Sizer (Beckman Coulter, Calif., USA). For particle size analysis, measurements were carried out at 25° C. at a light-scattering angle of 90° . The mean particle size and polydispersity index were recorded as shown in Table 1. The surface charge of the nanoparticles was analyzed by a Zetasizer, model HAS 3000 (Malvern, Worcestershire, England). There were no significant differences of the zeta potential values between modified and non-modified GPs.

TABLE 1

| | | | polydispersity |
|---|---|---|---|
| Particles | Size (nm) | Zeta (mV) | index |
| GP | 228.3 ± 71.0 | −9.3 ± 4.5 | 0.287 |
| GP-Av | 227.9 ± 26.0 | −9.4 ± 1.6 | 0.366 |
| GP-Av-bEGF | 241.9 ± 34.5 | −8.5 ± 0.8 | 0.489 |

*GP: gelatin nanoparticles; GP-Av: GP modified with NeutrAvidin^{FITC}; GP-Av-bEGF: GP-Av conjugated with biotunylated EGF.

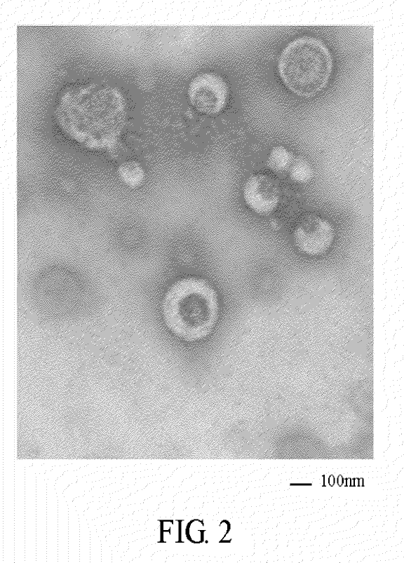

A photo of the GP-Av-bEGF nanoparticles was taken by transmission electron microscopy (TEM; Hitachi H-7500, Japan), showing a core-shell structure. Like FIG. 2, the core represented the gelatin nanoparticle, and it was shown that the complex of avidin and biotin surrounded the core.

### EXAMPLE 4

### Characterization of Aaerosol Particles

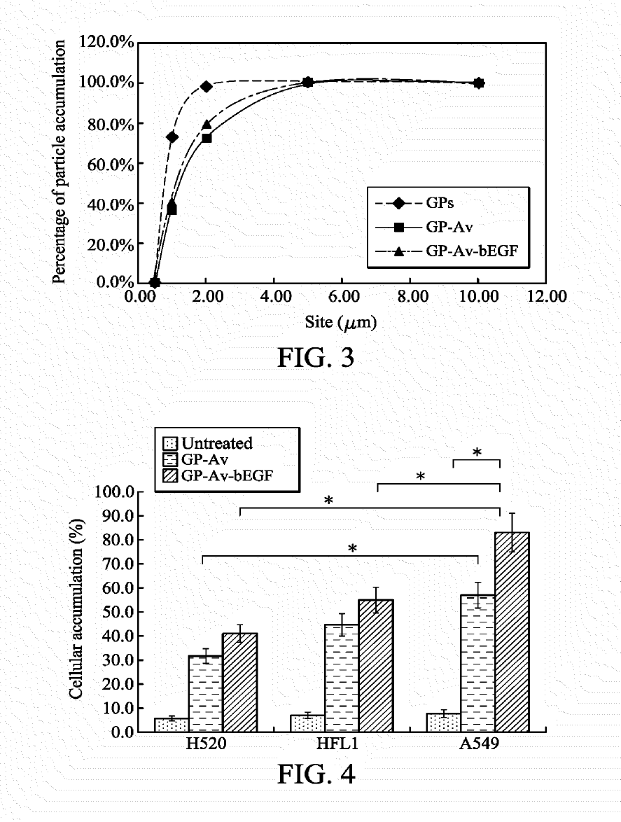

The size distribution of the nebulized GPs, GP-Av, and GP-Av-bEGF, each at a concentration of 100 μg/ml, was nebulized as an aerosol using a nebulizer (AP-100100; APEX, Taiwan) and analyzed using a DUST monitor (DUST-check portable dust monitor, model 1.108; Grimm Labortechnik Ltd., Germany), which is shown as FIG. 3.

The size distribution of the nebulized GPs, GP-Av, and GP-Av-bEGF all exhibited an extremely narrow distribution. More than 99% of all three GPs types were in the range of 0.1-5 μm in size. This represented the optimal size range for delivery via inhalation to prevent deposition of the particles on the air tract or escape through the upper respiratory track.

### EXAMPLE 5

### Determination of In Vitro Cellular Uptake of the GP-Av and GP-Av-bEGF

A549 (human lung adenocarcinoma cells), HFL1 (human normal fibroblast cells) and H520 (human squamous cell carcinoma) cells were seeded in a T-25 flask and grown to an 80% confluence. These cells were then respectively treated with modified and non-modified nanoparticles at a particle concentration of 100 μg/ml. After incubation for 3 hours, the cells were washed twice with PBS, trypsinized, centrifuged and resuspended in PBS buffer. Analysis of particle uptake was acquired using a Becton Dickson, FACScan flow cytometer system (Becton Dickinson, USA).

The result is shown in FIG. 4. The nanoparticles (GP-Av and GP-Av-bEGF) were highly accumulated on the A549 cell, wherein the GP-Av-bEGF showed higher accumulation than the GP-Av. The GP-Av-bEGF showed up to 81% cellular accumulation for the A549 cells due to highly EGFR expression on it. The values of FIG. 4 represent mean±SE, and n=3. A one-way ANOVA analysis was used, wherein p≦0.05.

### EXAMPLE 6

### In Vivo Aerosol Delivery of the GP-Av and GP-Av-bEGF

Male nude mice, 5-6 weeks of age, weighing above 20 g, were maintained under specific pathogen-free conditions and supplied with autoclaved mouse chow and water.

A549 human lung adenocarcinoma cells were cultured in T-75 flasks using a Ham's F12K medium supplemented with 10% fetal calf serum (FCS), 1% penicillin/streptomycin, and 1.5 g/l sodium bicarbonate. All cultures were incubated in 5% CO₂ at 37° C. Having obtained a sufficient cell number, the A549 cells (6×10⁶ cells suspended in 0.15 ml PBS) were injected into the tail veins of the male BALB/cAnN.Cg-Foxn1nu/CrlNarl nude mice, in order to induce pulmonary tissue tumors.

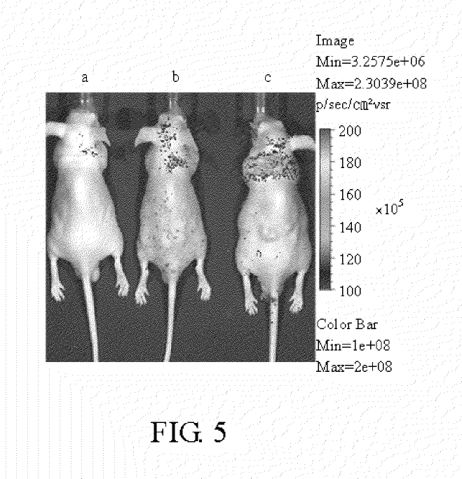

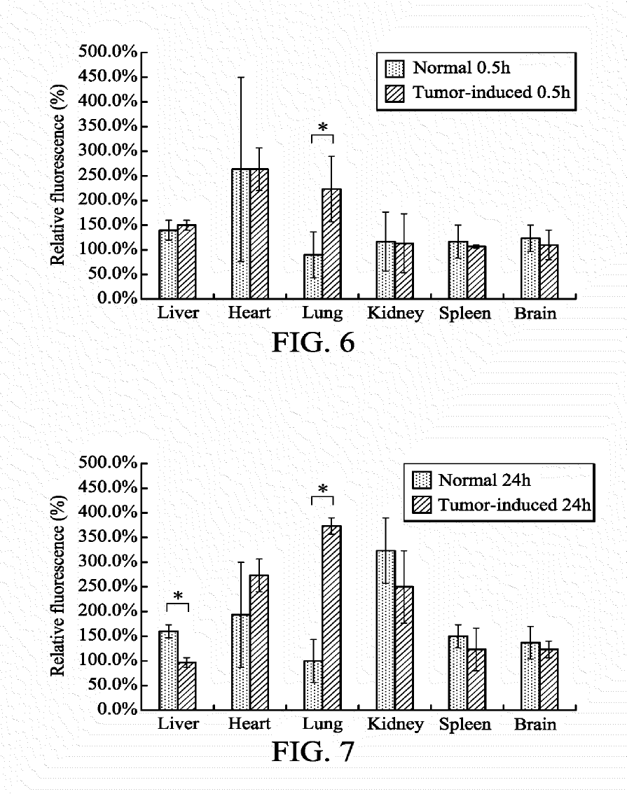

The targeting efficiency of the developed GP-Av-bEGF in vivo was evaluated by an in vivo imaging system (IVIS Imaging System 50; Xenogen, USA). Tumor-induced mice were divided into 3 groups and individually treated with PBS, GP-Av, or GP-Av-bEGF. The nanoparticle solution was nebulized as an aerosol and delivered to the mice through inhalation. The mice were exposed to the aerosol in a sealed plastic box. 5 ml of nanoparticle solution in the reservoir of the nebulizer was consumed within 30 min. The total deposited dose of the GP-Av-bEGF was 5 mg/kg/mouse for each exposure. Mouse images were obtained 1 day after aerosol delivery as shown in FIG. 5. The distribution of the developed GP-Av-bEGF in each organ was examined using the imaging system as described above. Normal mice and tumor-induced mice were all treated with the GP-Av-bEGF nanoparticles (n=4) using the same delivery process as described in the previous section. The mice were sacrificed at different time points (0.5 and 24 h). Lungs, heart, liver, kidneys, spleen, and brain were harvested and cleaned by PBS as shown in FIGS. 6 and 7. The relative intensity of fluorescence in each organ was equivalent to the concentration of fluorescent nanoparticles.

FIG. 5 shows the distribution of the nanoparticles in live mice 24 hours following aerosol delivery. Tumor-induced mice under gas anesthesia were used to monitor the particle distribution in real time. FIG. 5(a) is a PBS-treated mouse, FIG. 5(b) is a GP-Av-treated mouse, and FIG. 5(c) is a GP-Av-bEGF-treated mouse. Almost no fluorescence was detected in the PBS-treated mouse (FIG. 5(a)). The GP-Av-treated mouse (FIG. 5(b)) had median fluorescence distribution at the lung region. The mouse treated with the GP-Av-bEGF (FIG. 5(c)) showed the strongest fluorescence distribution at the lung region. Thus, GP-Av-bEGF was highly accumulated in the cancerous lungs after aerosol delivery.

FIG. 6 shows the relative fluorescence 0.5 hours after GP-Av-bEGF aerosol delivery. The GP-Av-bEGF was transported to each organ by exchanges between the pulmonary alveoli and capillary epithelial cells. A relatively high proportion of the GP-Av-
bEGF was accumulated in the lung of the tumor-induced mice both at 0.5 hours and 24 hours after treatment. As shown in FIG. 7, accumulation of the GP-Av-bEGF 24 hours after treatment was significantly higher than accumulation of the GP-Av-bEGF 0.5 hours after treatment. The result showed that the GP-Av-bEGF accumulated in the tumor induced lung. Other organs showed no significant difference between the tumor-induced lungs or normal lung groups. Thus, the accumulation of the GP-Av-bEGF in the cancerous lungs was caused by the binding of the EGF to the EGFR cancerous cells, not only for the local administration of inhalation.

### EXAMPLE 7

### Synthesis of Nanocomplexes of Cisplatin Encapsulated by GP-Av-bEGF

5 mg of Cisplatin (CDDP, H₆Cl₂N₂Pt) was added into a 5 ml solution of GPs produced from Example 1, and reacted at 37° C. for 24 hours. Thus, forming CDDP capsulated by GP (GP-CDDP).

Following the processes of Examples 3 and 4, GP-CDDP with NeutrAvidin^{FITC} linked on its surface was formed. After conjugated with bEGF, the nanocomplex of Cisplatin is encapsulated by GP-Av-bEGF and forms GP-CDDP-bEGF.

### EXAMPLE 8

### CDDP In Vitro Anticancer Activity Assay

A549 cells were placed in a 96-well culture plat at a density of 5×10⁴ cells per well in 0.1 ml of growth medium and cultured for 24 hours. The cells were respectfully reseeded with free CDDP, GP-CDDP and GP-CDDP-bEGF at different concentrations (CDDP concentration from 1.5˜250 μM) and co-incubated for 3 days. Each group was analyzed by MTT (working concentration 0.5 mg/ml) as shown in Table 2. The percentage of cell viability is illustrated as the ratio of the absorbance of treated cells relative to the absorbance of non-treated cells.

TABLE 2
IC₅₀ of A549 cells with treatment of CDDP,
GP-CDDP and GP-CDDP-bEGF

IC₅₀ Value

| Formula | 48 hours | 72 hours |
|---|---|---|
| CDDP | 13.62 | 5.20 |
| GP-CDDP | 31.00 | 20.78 |
| GP-CDDP-bEGF | 4.23 | 3.95 |

According to Table 2, A549 cells treated with GP-CDDP-bEGF for 48 or 72 hours exhibited stronger cytotoxicity than those treated with GP-CDDP. The GP-CDDP-bEGF accumulated at the cell line of A549 and killed the cells with a minimum dose as it may be highly specific to A549 cells. The IC₅₀ value for cells treated with GP-CDDP for 48 hours was significantly different from those treated for 72 hours, which showed slow release of CDDP from GP.

### EXAMPLE 9

### In Vivo Distribution of CDDP, GP-CDDP and GP-CDDP-bEGF With Inhalation

Male CB-17/lcrCrl-severe combined immunodeficient (SCID)-bg mice, 5-6 weeks of age, weighing above 20 g, were maintained under specific pathogen-free conditions and supplied with autoclaved mouse chow and water.

A549 human lung adenocarcinoma cells were cultured in T-75 flasks using a Ham's F12K medium supplemented with 10% fetal calf serum (FCS), 1% penicillin/streptomycin, and 1.5 g/l sodium bicarbonate. All cultures were incubated in 5% CO₂ at 37° C. Having obtained a sufficient cell number, the A549 cells (6×10⁶ cells suspended in 0.15 ml PBS) were injected into the tail vein of the male SCID mice, in order to induce pulmonary tissue tumors.

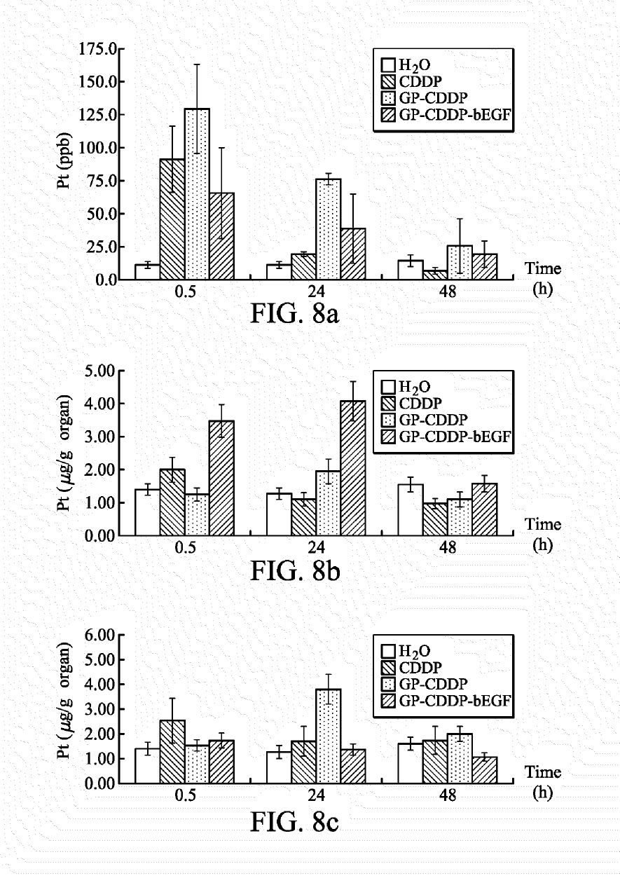

The mice were put in a sealed plastic box which was nebulized with 5 ml of Cisplatin-GPs solution (GP-CDDP, GP-CDDP-bEGF, free CDDP) and H₂O (control) within 30 min. The total deposition dose of cisplatin during the inhalation period was theoretically 12 mg/kg per mice. CDDP (Pt) concentration in blood, lungs and kidneys were examined. At different time intervals (0.5, 24 and 48 hours), three mice were sacrificed and their blood were directly taken from the heart with a heparinized syringe. The lungs and kidneys were sampled to measure the Pt concentration. Organs were removed and washed by PBS before assay. The organs were soaked in 1 ml of a 70% nitric acid solution overnight and then digested for 2 hours at 90° C. After that, 5 ml of deionized water was added to form the final volume as 5 ml, and was filtered in a 0.22 μm PVDF filter. The solutions were assayed by an inductively coupled plasma atomic emission spectroscopy (ICP-AES) to confirm the Pt content in organs as shown in FIGS. 8(a)˜(c).

FIG. 8(a) shows that the CDDP treated group had high Pt concentrations in plasma during the initial half hour, but concentrations decreased quickly after 24 and 48 hours later. The serum Pt level of the GP-CDDP-bEGF decreased slowly from 0.5 to 48 hours and maintained a stable Pt concentration during the time of observation. FIG. 8(b) shows Pt concentration in the lungs at 0.5 to 48 hours. The GP-CDDP-bEGF exhibited the highest Pt concentrations within 0.5 and 24 hours after administration. Thus, the cancer cells with EGFR overexpression entrapped the EGF modified nanoparticles quickly to cause a higher amount of Pt to accumulate in the lungs. The GP-CDDP, without EGF modification, did not accumulate in the lungs, and showed similar concentration changes like the free CDDP. From FIGS. 8(a) to (c), it was shown that tissue concentrations of the Pt in the serum, lungs and kidney were lower for free CDDP than that for CDDP with carriers. Thus, free drugs were quickly cleared in the body.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A pharmaceutical composition for inhalation delivery, comprising drugs and a gelatin nanoparticle encapsulating the drugs to form a drug-gelatin nanocomplex, wherein the surface of the gelatin nanoparticle is modified with cell-targeting molecules.

2. The pharmaceutical composition for inhalation delivery as claimed in claim 1, wherein the cell-targeting molecules comprise epidermal growth factor (EGF), anti-EGR receptor (EGFR) antibodies, anti-EGFR peptide, EGFR inhibitors; anti-human epidermal growth factor receptor 2 (HER2) antibody, anti-HER2 peptide, HER2 inhibitor; folic acid, folic acid analogy; vascular epithelial growth factor (VEGF), anti-VEGF receptor (VEGFR) antibodies, anti-VEGFR peptide, VEGFR inhibitor; or insulin-like growth factors (IGFs), anti-IGFs receptor (IGFR) antibody, anti-IGFR peptide or IGFR inhibitor.

3. The pharmaceutical composition for inhalation delivery as claimed in claim 1, wherein the modification comprises a linking of specific molecules.

4. The pharmaceutical composition for inhalation delivery as claimed in claim 3, wherein the linking of the specific molecules comprises the linking of avidin and biotin, antigen and antibodies, or cell markers.

5. The pharmaceutical composition for inhalation delivery as claimed in claim 4, wherein the specific molecule and the cell-targeting molecules comprise a molar ratio of 4:1˜30:1.

6. The pharmaceutical composition for inhalation delivery as claimed in claim 4, wherein the specific molecules further comprises a fluorescent marker.

7. The pharmaceutical composition for inhalation delivery as claimed in claim 1, wherein the gelatin nanoparticle further comprises a cross linker.

8. The pharmaceutical composition for inhalation delivery as claimed in claim 7, wherein the cross linker comprises formaldehyde, glutaraldehyde, genipin, or diadehyde starch.

9. The pharmaceutical composition for inhalation delivery as claimed in claim 8, wherein the cross linker comprises 0.01-0.1% in weight based on the weight of the gelatin.

10. The pharmaceutical composition for inhalation delivery as claimed in claim 8, wherein the cross-linking degree between the cross linker and the gelatin is 1˜20%.

11. The pharmaceutical composition for inhalation delivery as claimed in claim 1, wherein the drugs comprise medicaments for respiratory tracts, lungs or anticancer treatments.

12. The pharmaceutical composition for inhalation delivery as claimed in claim 11, wherein the drugs comprises 1-60% in weight based on the weight of the gelatin.

13. The pharmaceutical composition for inhalation delivery as claimed in claim 11, wherein the drugs comprises 5-Fluorouracil (5-FU), Cisplatin, Carboplatin, Gemcitabine, Vinorelbine, Paclitaxel, Docetaxel, Doxorubin, steroid, Albuterol, Salbutamol, Salmeterol, or Ipratropium.

14. The pharmaceutical composition for inhalation delivery as claimed in claim 1, wherein the pharmaceutical composition is packed in a nebulizer.

15. The pharmaceutical composition for inhalation delivery as claimed in claim 14, wherein the pharmaceutical composition is nebulized as aerosol droplets with 0.1-5 μm in radius.

16. The pharmaceutical composition for inhalation delivery as claimed in claim 1, further comprises a pharmaceutically acceptable carrier and/or additives.

17. A method for fabricating a pharmaceutical composition for inhalation delivery, comprising:
providing a drug;
adding the drug into a gelatin solution to form a gelatin nanoparticle encapsulating drug; and
linking cell-targeting molecules on the surface of the gelatin nanoparticle for modification,
wherein the drug and the gelatin nanoparticle form a drug-gelatin nanocomplex.

18. The method for fabricating a pharmaceutical composition for inhalation delivery as claimed in claim 17, wherein the cell-targeting molecules comprise epidermal growth factor (EGF), anti-EGR receptor (EGFR) antibodies, anti-EGFR peptide, EGFR inhibitors; anti-human epidermal growth factor receptor 2 (HER2) antibody, anti-HER2 peptide, HER2 inhibitor; folic acid, folic acid analogy; vascular epithelial growth factor (VEGF), anti-VEGF receptor (VEGFR) antibodies, anti-VEGFR peptide, VEGFR inhibitor; or insulin-like growth factors (IGFs), anti-IGFs receptor (IGFR) antibody, anti-IGFR peptide or IGFR inhibitor.

19. The method for fabricating a pharmaceutical composition for inhalation delivery as claimed in claim 17, wherein the modification comprises a linking of specific molecules.

20. The method for fabricating a pharmaceutical composition for inhalation delivery as claimed in claim 19, wherein the linking of the specific molecules comprises the linking of avidin and biotin, antigen and antibodies, or cell markers.

21. The method for fabricating a pharmaceutical composition for inhalation delivery as claimed in claim 20, wherein the specific molecule and the cell-targeting molecules comprise a molar ratio of 4:1˜30:1.

22. The method for fabricating a pharmaceutical composition for inhalation delivery as claimed in claim 20, wherein the specific molecules further comprises a fluorescent marker.

23. The method for fabricating a pharmaceutical composition for inhalation delivery as claimed in claim 17, wherein the gelatin solution comprises a concentration of 0.1-10% (weight-to-volume ratio) in deionized water.

24. The method for fabricating a pharmaceutical composition for inhalation delivery as claimed in claim 17, wherein the gelatin nanoparticle further comprises a cross linker.

25. The method for fabricating a pharmaceutical composition for inhalation delivery as claimed in claim 24, wherein the cross linker comprises formaldehyde, glutaraldehyde, genipin, or diadehyde starch.

26. The method for fabricating a pharmaceutical composition for inhalation delivery as claimed in claim 25, wherein the cross linker comprises 0.01-0.1% in weight based on the weight of the gelatin.

27. The method for fabricating a pharmaceutical composition for inhalation delivery as claimed in claim 25, wherein the cross-linking degree of the cross linker and the gelatin is 1-20%.

28. The method for fabricating a pharmaceutical composition for inhalation delivery as claimed in claim 17, wherein the drugs comprise medicaments for respiratory tracts, lungs or anticancer treatments.

29. The method for fabricating a pharmaceutical composition for inhalation delivery as claimed in claim 28, wherein the drugs is 1-60% in weight based on the weight of the gelatin.

30. The method for fabricating a pharmaceutical composition for inhalation delivery as claimed in claim 28, wherein the drugs comprises 5-Fluorouracil (5-FU), Cisplatin, Carboplatin, Gemcitabine, Vinorelbine, Paclitaxel, Docetaxel, Doxorubin, steroid, Albuterol, Salbutamol, Salmeterol, or Ipratropium.

31. The method for fabricating a pharmaceutical composition for inhalation delivery as claimed in claim 17, wherein the pharmaceutical composition is nebulized.

32. The method for fabricating a pharmaceutical composition for inhalation delivery as claimed in claim 31, wherein the pharmaceutical composition is nebulized as aerosol droplets with 0.1-5 μm in radius.
